(19) United States
(12) Patent Application Publication
(10) Pub. No.: US 2002/0038092 A1
(43) Pub. Date: Mar. 28, 2002

(54) Capacitively coupled electrode system for sensing voltage potentials at the surface of tissue

(76) Inventors: Thomas G. Stanaland, Winchester, CA (US); Gaetan Chevalier, Encinitas, CA (US)

Correspondence Address: STRADLING YOCCA CARLSON & RAUTH, IP Department, P.O. Box 7680, 660 Newport Center Drive, Suite 1600, Newport Beach, CA, 92660-6441, US

(21) Appl. No.: 09/882,229

(22) Filed: Jun. 15, 2001

Related U.S. Application Data
(63) Continuation-in-part of application No. 09/636,541, filed on Aug. 10, 2000.

(51) Int. Cl. A61B 5/0408

(52) U.S. Cl. 600/509

(57) ABSTRACT

An electrical activity sensor for sensing and reproducing electrical potentials at the surface of a test item, such as a human being, has an electrode configured to be capacitively coupled to the test item and a detection circuit coupled to the electrode. The detection circuit compensates for capacitive coupling of the electrode. The capacitively coupled electrode and the detection circuit capacitance cooperate to mitigate a need for conductively coupling the electrode to the test subject.

### RELATED APPLICATION

This patent application is a Continuation-In-Part patent application of U.S. Ser. No. 09/636,541, filed on Aug. 10, 2000 and entitled CAPACITIVELY COUPLED ELECTRODE SYSTEM WITH VARIABLE CAPACITANCE FOR SENSING POTENTIALS AT THE SURFACE OF TISSUE, the entire contents of which are hereby expressly incorporated by reference.

### FIELD OF THE INVENTION

The present invention relates generally to medical electrical sensing devices such as electroencephalograph (EEG), electromyograph (EMG), electrocardiograph (EKG) and galvanic skin response (GRS) devices. The present invention relates more particularly to a capacitively coupled electrode system including a capacitively coupled electrode for sensing electric potentials at the surface of living tissue, as well as at the surface of any other desired item.

### BACKGROUND OF THE INVENTION

The use of electrodes for sensing electrical activity at the surface of living tissue, such as during the performance of an electroencephalograph (EEG), an electromyograph (EMG), an electrocardiograph (EKG) or a galvanic skin response (GSR) procedure is well-known. Such contemporary electrodes provide resistive coupling to the test subject, so as to facilitate the monitoring of electrical activity therein.

Although such contemporary resistively coupled electrodes are generally suitable for the intended purposes, resistively coupled electrodes do possess inherent deficiencies which detract from their utility. For example, conductive gels, paste or adhesives are typically utilized when performing an EEG, EMG, EKG or GSR procedure so as to assure the necessary ohmic conduction, i.e., good electrical contact, between such contemporary electrodes and the test subject. The conductive gel, paste or adhesive is generally applied to the contemporary electrode and/or test subject to eliminate non-conductive air gaps there between.

Those skilled in the art will appreciate that the use of such conductive paste, gel and/or adhesive can be very messy, particularly when the test subject has thick hair at the site where the electrode is to be placed. The presence of such hair may necessitate shaving of the site in order to assure adequate electrical contact between the electrode and the skin. The presence of even a very small gap between the contemporary electrode and the surface of the skin, such as that which may be caused by hair, tends to adversely affect the monitoring of electrical activity and is therefore undesirable.

For example, it is common practice for EEG or neurofeedback practitioners to ensure that the resistance of the skin of the test subject's scalp is less than 5k ohms before proceeding with an EEG procedure. In order to obtain such low skin resistance upon the scalp, the neurofeedback practitioner must often utilize an abrasive paste with which the skin of the scalp is rubbed quite intensely. As one may imagine, such intense abrasion of the scalp may cause undesirable pain and may even result in bleeding.

Because of the possible pain and lengthy skin preparation process involved in such EEG procedures, a test subject may postpone or even cancel EEG procedures and may even choose to forego further EEG assessment all together.

The use of such contemporary conductively coupled electrodes may necessitate that the head of the test subject be shaved when, for example, it is necessary to access damage caused by a head injury or brain tumor. During neurofeedback and/or sleep studies, the test subject may be required to wear a helmet or cap within which contemporary conductively coupled electrodes are mounted. Such helmets or caps help to ensure the stability of the position of the conductive electrodes when the electrodes must remain in place for an extended period of time. When such a helmet or cap is utilized, then the neurofeedback practitioner is required to inject a conducting gel or paste through the helmet or cap utilizing a syringe. Occasionally, the neurofeedback or EEG recording practitioner cannot obtain good conduction at a particular site such as excessive conducting gel from one site running together with gel from another site and the helmet or cap must be removed so that the problem affecting such conduction may be addressed.

Such repeated application and removal of the helmet is undesirable and time consuming.

The performance and reliability of such contemporary conductively coupled electrodes is degraded by the presence of hair, as well as any other foreign substances (dried blood, dirt, etc.), which might be present upon the skin at the desired sight of the electrode. This is a particular problem when a patient in an emergency room, for example, is suspected of being in cardiac arrest and the doctor needs to perform an EKG measurement as soon as possible.

Hair and other such foreign matter is particularly troublesome in emergency situations, where it may not be possible to shave or clean the affected area. For example, a portable EKG monitor, which may be used to provide medical information to medical personnel at the remote site or may be used to control a defibrilator, must be operated immediately, i.e., without time to shave or clean the sites where electrodes are to be applied to the test subject.

The performance of such a contemporary electrode is degraded by the presence of hair and other materials because hair and other materials tend to physically separate the electrode from the test subject's skin, thereby increasing the resistance of the coupling and degrading the electrical contact between the electrode and the test subject. It is possible that such hair and other material may interfere with the performance of the electrodes sufficiently to render the electrode ineffective in performing its desired function.

In view of the foregoing, it is desirable to provide an electrode suitable for use in EEG, EMG, EKG, and GSR procedures and the like and which does not require conductive coupling to the test subject and is therefore not substantially sensitive to the presence of hair and/or other materials which degrade the performance of contemporary conductively coupled electrodes.

### SUMMARY OF THE INVENTION

The present invention specifically addresses and alleviates the above-mentioned deficiencies associated with the prior art. More particularly, the present invention comprises an electrical activity sensor which comprises an electrode configured to be capacitively coupled to an object being monitored and a detection circuit configured to mitigate a capacitive effect of the capacitively coupled electrode. The capacitively coupled electrode and the detection circuit cooperate to mitigate the prior art need for conductively coupling the electrode to the test subject.

These, as well as other advantages of the present invention, will be more apparent from the following description and the drawings. It is understood that changes in the specific structure shown and described may be made within the scope of the claims without departing from the spirit of the invention.

### DESCRIPTION OF THE DRAWINGS

FIG. 1 is a block diagram showing a first embodiment of the system for sensing and reproducing electrical signals according to the present invention, wherein a variable capacitance is utilized;

FIGS. 2A and 2B show one example of a capacitively coupled electrode suitable for use in the first, second and third embodiments of the present invention;

FIG. 3 shows one example of a variable capacitance device formed according to the first embodiment of the present invention;

FIG. 4 is a simplified electrical schematic (as used in a circuit simulation) showing the system for sensing and reproducing electrical signals according to the first embodiment of the present invention;

FIG. 5 is a graph showing an exemplary input signal, (V_{IN}) of FIG. 4, as used in a simulation of the first embodiment of the present invention;

FIG. 6 is a graph showing an exemplary output voltage, (Vₒᵤₜ) of FIG. 4, according to the first embodiment of the simulation;

FIG. 7 is a Bode diagram showing output voltage and phase versus frequency according to the simulation of the circuit of FIG. 4;

FIG. 8 is a block diagram which is common to both second and third embodiments of the present invention, wherein a detection circuit compensates for capacitive effects of the capacitively coupled electrode and differentiates a signal provided by the a capacitively coupled electrode and an integration circuit integrates the differentiated signal;

FIG. 9 is an electrical schematic showing the detection circuit according to a second embodiment of the present invention, wherein a variable capacitance is not utilized;

FIG. 10 is a chart showing the input voltage versus time used in a simulation of the circuit of FIG. 9, so as to provide the results show in FIGS. 11, 12 and 13;

FIG. 11 is a chart showing amplitude of the output voltage versus time for the detection circuit of FIG. 9;

FIG. 12 is a chart showing the variation of phase versus frequency for the detection circuit shown in FIG. 9;

FIG. 13 is a chart showing the frequency response of the detection circuit of FIG. 9;

FIG. 14 is an electrical schematic showing the detection circuit according to a third embodiment of the present invention, wherein a variable capacitance is not required;

FIG. 15 is a chart showing the input voltage versus time used in a simulation of the circuit of FIG. 14, so as to provide the results show in FIGS. 16, 17 and 18;

FIG. 16 is a chart showing amplitude of the output voltage versus time for the detection circuit of FIG. 14;

FIG. 17 is a chart showing the variation of phase versus frequency for the detection circuit shown in FIG. 14; and

FIG. 18 is a chart showing the frequency response of the detection circuit of FIG. 14.

This exemplary capacitively coupled electrode is suitable for use with the first, second and third embodiments of the present invention.

### DETAILED DESCRIPTION OF THE INVENTION

The detailed description set forth below in connection with the appended drawings is intended as a description of the presently preferred embodiments of the invention and is not intended to represent the only forms in which the present invention may be constructed or utilized. The description sets forth the functions and the sequence of steps for constructing and operating the invention in connection with each of the illustrated embodiments. It is to be understood, however, that the same or equivalent functions and sequences may be accomplished by different embodiments that are also intended to be encompassed within the spirit and scope of the invention.

The electrode of the present invention comprises a conductive member and a dielectric member which is configured to inhibit contact of the conductive member with the test subject. The conductive member is preferably configured as a disk and the dielectric cover preferably substantially surrounds the disk-shaped conductive member.

The electrode is configured to be capacitively coupled to living tissue. Further, the electrode is preferably configured to be capacitively coupled to a mammal, such as a human being. Those skilled in the art will appreciate that the capacitively coupled electrode of the present invention is suitable for use in various different applications, such as veterinary applications. Indeed, the capacitively coupled electrode of the present invention may be utilized to monitor electrical activity at the surface of non-living or non-biological material.

According to one aspect of the present invention, the electrode comprises a copper member generally configured as a disk, a dielectric cover substantially surrounding the conductive member and a cap comprised of an insulator which cooperates with a dielectric cover to generally enclose the copper member. At least one conductive lead is coupled to the copper member and extends through the cap, so as to facilitate electrical communication of the electrode with support circuitry, as discussed in detail below.

According to a first embodiment of the present invention, a variable capacitance is utilized as discussed in detail below. According to the second and third embodiments of the present invention, no variable capacitance is utilized and a detection circuit compensates for the capacitive effect provided by the capacitively coupled electrode.

Thus, according to the second and third embodiments of the present invention, a detection circuit is coupled to receive an output of the capacitively coupled electrode and to mitigate a capacitive effect of the capacitively coupled electrode. That is, the detection circuit tends to compensate for differences in the output of the electrode cause by the electrode being capacitively coupled, rather than directly electrically coupled. Further, according to one aspect of the present invention, the detection circuit differentiates (in the mathematical sense) the input signal. Thus, the detection circuit provides a signal which is representative of the input signal at the surface of the living tissue or test item, as discussed in detail below.

According to the second and third embodiments of the present invention, an amplifier is coupled so as to amplify an output of the detection circuit. The amplifier preferably comprises a differential amplifier, preferably a variable gain differential amplifier. The differential amplifier has two type of gains: a frequency dependent gain to adjust for the frequency dependent attenuation of the electrode system; and an adjustable frequency independent gain to ensure that the output signal simulate the input signal from the test item. In this manner, adjustments may be made as to compensate for inconsistencies in the electrical components of the electrode system of the present invention, as well as in the efficiency of coupling of the electrode to the test subject. Further, the variable gain amplifier may be adjusted as to amplify the output of the detection circuit in a manner which facilitates provision of an output which generally mimics an output of an EEG electrode, an EKG electrode, an EMG electrode, or a GSR electrode.

According to the second and third embodiments of the present invention, an integration circuit is coupled to the amplification circuit so as to receive an output of the amplification circuit and so as to integrate (in the mathematical sense) the output of the amplification circuit. Integration of the output of the amplification circuit tends to cancel the differentiation introduced by the detection circuit.

An output circuit is coupled to the integration circuit so as to define an output impedance. The output impedance may be selected so as to generally mimic the output impedance of an EEG electrode, an EMG electrode, an EKG electrode or a GSR electrode.

The capacitively coupled electrode system of the present invention further comprises a reference electrode which provides a reference to the detection circuit. The capacitively coupled electrode system of the present invention further comprises a ground electrode coupled to an electrically conductive box designed to enclose the electrical components comprising the capacitively coupled electrode of this invention as explained in details below. The reference electrode and the ground electrode function in a manner analogous to reference and ground electrodes of contemporary EEG, EMG, EKG and/or GSR systems.

Thus, according to the present invention, an electrical activity sensor system comprising a capacitively coupled electrode which is electrically coupled to a detection circuit and an integration circuit utilizes displacement current to sense electrical activity at the surface of a test subject.

The present invention is generally described herein as being particularly suited for use in medical applications such as an electroencephalograph (EEG), an electromyograph (EMG), an electrocardiograph (EKG) or a galvanic skin response (GSR) device. However, such description is by way of illustration only, and not by way of limitation. Indeed, the present invention may find applications in various unrelated fields. Thus, the present invention may be utilized to capacitively couple an electrode to any desired test items, either living, dead, inanimate, organic or inorganic. Indeed, the present invention may be utilized to measure electrical activity in any desired test item for which such capacitive coupling is appropriate.

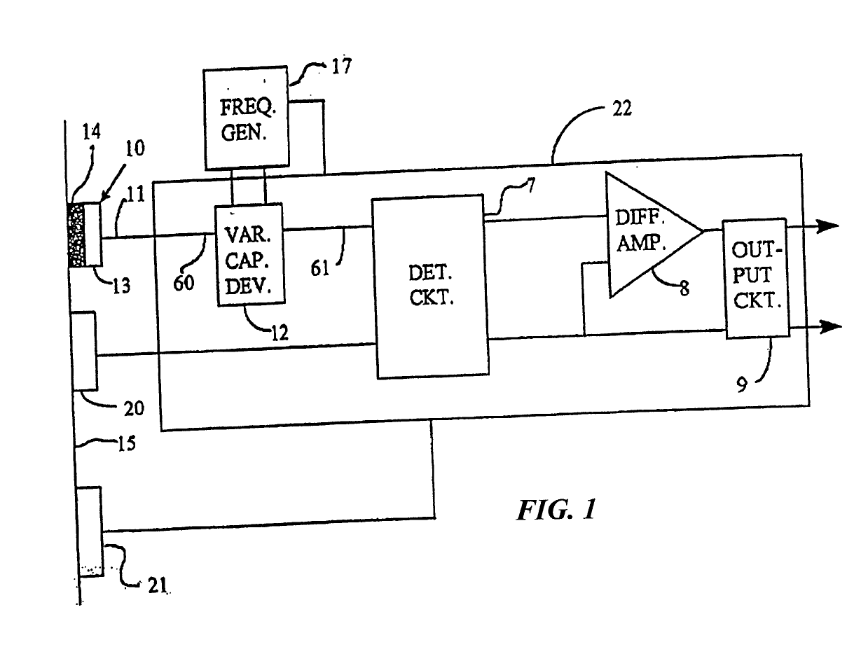

FIGS. 1 and 3-7 illustrate the first embodiment of the capacitively coupled electrode system of the present invention and data obtained from computerized simulations there.

Referring now to FIG. 1, an exemplary first embodiment of the capacitively coupled electrode system of the present invention generally comprises a capacitively coupled electrode 10 which is in electrical communication with a variable capacitance device 12. A detection circuit 7 receives the output of the variable capacitance device 12 and conditions the output of the variable capacitance device 12 as described below. An amplifier 8 receives the output of the detection circuit 7 and amplifies or attenuates that output as described below. An output circuit 9 is in electrical communication with the amplifier 8 so as to receive the output of the amplifier 8 and determine an output impedance of the capacitively coupled electrode system.

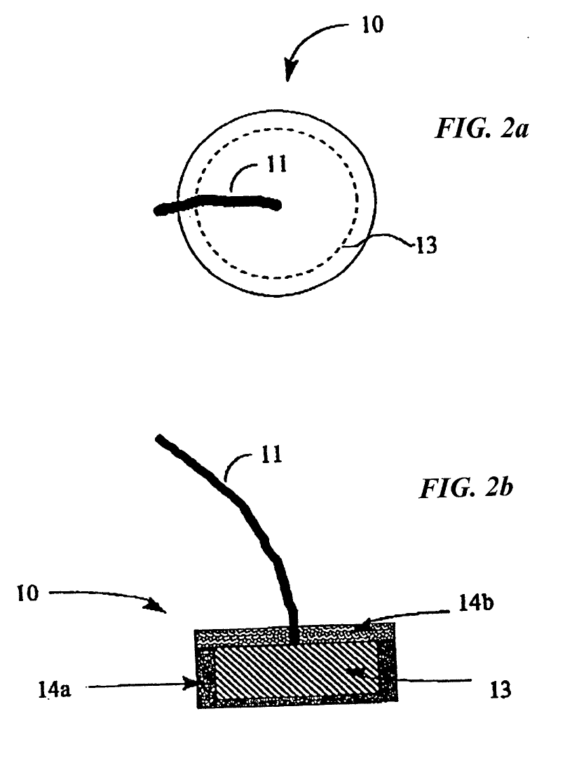

The capacitively coupled electrode 10 (better shown in FIGS. 2A and 2B) generally comprises a conductive member 13 and a non-conductive member 14. The conductive member 13 defines a capacitor plate which facilitates the sensing of electrical activity within a test item or subject 15. The non-conductive member 14 electrically isolates the conductive member 13 from the test subject 15.

Thus, the capacitively coupled electrode 10 is capacitively coupled, rather than conductively coupled, to the test subject 15. Because of this capacitive coupling, displacement current may be utilized to effect sensing of electrical signals at the test subject. As discussed above, such capacitive coupling provides substantial advantages in eliminating the need for good electrical contact between the electrode 10 and the test subject 15.

Various different configurations of the capacitively coupled electrode 10 are contemplated.

For example, the conductive member 13 of the capacitively coupled electrode 10 may be electrically isolated from the test subject 15 via a non-conducting layer 14 formed upon one surface thereof only, as shown in FIG. 1. Alternatively, the conductive member 13 of the capacitively coupled electrode 10 may be substantially encapsulated within a non-conductor as shown in FIGS. 2A and 2B. Substantially encapsulating the conductive member 13 within a non-conducting layer 14 mitigates the likelihood of the conductive member 13 inadvertently contacting the test subject, and thus degrading the performance of the capacitively coupled electrode of the present invention.

The variable capacitance device 12 is generally defined by a capacitor, the capacitance of which can be varied, preferably in a controlled fashion. Thus, the plate area of the capacitor, the spacing between the plates of the capacitor and/or the dielectric constant of the capacitor of the variable capacitance device 12 may be varied. According to the preferred embodiment of the present invention, a frequency source 17 provides a frequency input to the variable capacitance device 12, so as to effect varying of the capacitance of the variable capacitance device 12, as desired. The detection circuit 7 conditions the output of the variable capacitance device 12, so as to make the signal suitable for amplification by the amplifier 8.

The frequency generator may comprise a commercially available frequency generator or, alternatively, may comprise a frequency generator built specifically for use with the variable capacitance device 12. In either instance, the frequency source 17 is preferably electrically grounded to the electrical box 22 to provide protection to the remainder of the capacitively coupled electrode system, so as to mitigate any likelihood of an undesirable electrical shock to the patient.

The frequency generator 17 may optionally be disposed within the box depending on its size. In case it is out of the box 22, the frequency generator 17 should be grounded to the box 22. The role of the ground electrode 21 connected to the box 22 is to protect the test item from any possible electrical shocks that could be generated by the electrical components of the electrode circuit. This type of grounding using a box with electronic components inside it to protect a test item from possible electric shocks is standard procedure in the industry of EEG systems.

Referring now to FIGS. 2A and 2B, an exemplary capacitively coupled electrode is shown. This exemplary capacitively coupled electrode may be utilized with either the first, second or third embodiment of the present invention. With particular reference to 2A, the exemplary capacitively coupled electrode 10 is preferably generally circular in configuration, so as to define a disk. However, those skilled in the art will appreciate that various other configurations of the capacitively coupled electrode 10 are likewise suitable. A conductive conduit or lead 11 extends from the capacitively coupled electrode 10 so as to facilitate electrical communication with the variable capacitance device 12 (FIG. 1). Lead 11 is electrically coupled to the conductive member 13 of the capacitively coupled electrode 10.

With particular reference to FIG. 2B, the conductive member 13 of the capacitively coupled electrode 10 may, if desired, be generally completely encapsulated within a non-conductive housing so as to mitigate problems associated with inadvertent contact of the conductive member 13 with the test subject 15 (FIG. 1). As shown in FIG. 2B, a dielectric material contacting portion 14A generally surrounds most of the conductive member 13 and a dielectric cap 14B generally covers the remaining portion of the conductive member 13. The lead 11 is insulated. Thus, inadvertent electrical contact with the test subject of the lead 11 and/or the conductive member 13 is substantially inhibited.

The conductive member 13 is preferably comprised of copper. However, those skilled in the art will appreciate that various other conductive substances, particularly metals, are likewise suitable. The non-conductive housing 14A, 14B, may be comprised of any suitable, preferably biologically compatible, dielectric material such as plastic, rubber, epoxy, etc.

The conductive member 13 is preferably about 1 cm diameter but the dimension can be changed to fit the needs of the clinical or other setting. The shape of the electrode can also be varied as desired. Thus, the electrode can be sized and configured so as to be suitable for the test item or test subject. The wire or lead 11 itself is preferably a part of the electrode of this invention. The front side of the electrode (the active side, which is the side in contact with the body or almost in contact with the body if there is something preventing direct contact, such as body hairs) is covered with a thin layer of a material with a high dielectric constant such as Teflon or a ceramic. Such materials have a high dielectric constant, which is ideal for this application. The backside of the electrode is protected by an insulating material.

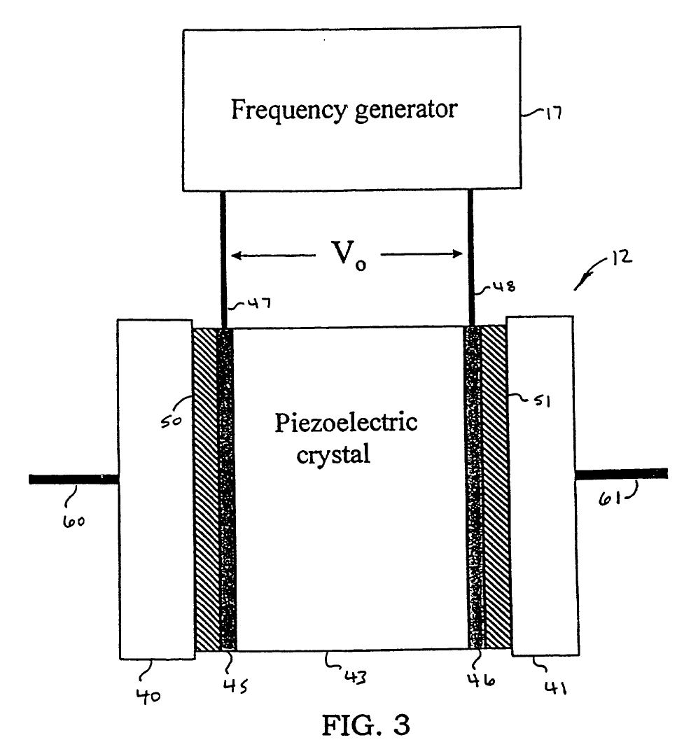

Referring now to FIG. 3, an exemplary embodiment of the variable capacitance device 12 comprises first 40 and second 41 conductive plates which define a capacitor. The first 40 and second 41 conductive plates are movable with respect to one another, such that the distance there between is easily varied. A piezoelectric crystal 43 or the like is disposed intermediate the first 40 and second 41 conductive plates so as to affect movement of the first 40 and second 41 conductive plates relative to one another. The frequency source 17 is coupled so as to provide a voltage across the piezoelectric crystal 43 in order to effect compression and expansion of the piezoelectric crystal 43, thus varying the distance between the first 40 and second 41 plates of the capacitor defined thereby. In this manner, the frequency source 17 controls the capacitance of the variable capacitance device 12

Preferably, conductive coatings 45 and 46 are applied to the piezoelectric crystal 43, so as to facilitate desired electrical contact with the leads 47 and 48, which provide electrical communication between the piezoelectric crystal 43 and the frequency source 17.

Preferably, epoxy layers 50 and 51 facilitate mechanical attachment of the piezoelectric crystal 43 (via the conductive coatings 45 and 46) to the conductive plates 40 and 41. Those skilled in the art will appreciate that various other means for fastening the conductive plates 40 and 41 to the piezoelectric crystal are likewise suitable. For example, the conductive plates 40 and 41 may be held in place with respect to the piezoelectric crystal 43 via the use of fasteners such as screws, preferably in combination with spring washers, such as Belville washers, which pass through the conductive plates 40 and 41 and the piezoelectric crystal 43. As a further alternative, spring clips may be utilized to bias the conductive plates 40 and 41 toward the piezoelectric crystal 43.

Lead 60 facilitates electrical communication of the first plate 40 with the capacitively coupled electrode 10. Similarly, lead 61 facilitates electrical communication of the second plate 41 with the detection circuit 7.

The variable frequency source 17, such as a commercially available frequency generator, generates a sinusoidal voltage Vₒ=Vₒ′ sin ω′t. This voltage is applied to a piezoelectric crystal 43 placed between the two plates 40 and 41 of the parallel plate variable capacitor. The voltage Vₒ is transmitted to the crystal 43 through conduction plates 45 and 46, which cover the side surfaces of the piezoelectric crystal 43. The piezoelectric crystal 43 is attached to the two plates in such a manner that the voltage Vₒ cannot leak to the parallel plates 40 and 41 of the variable capacitor 12 (in which case this voltage Vₒ would interfere with the potential of the body). This is preferably accomplished by attaching the crystal of the plates 40 and 41 using an epoxy having a high dielectric constant. The applied voltage Vₒ modify the thickness of the piezoelectric crystal in a sinusoidal manner. This results in a sinusoidal modulation of the distance between the plates of the capacitor d=d₀(1+δ sin ω′t) where d₀ is the distance between the two plates of the parallel plate capacitor when there is no voltage applied to the piezoelectric crystal, i.e., V₀=0. The parameter δ is a modulation factor dependent in a complex manner on the amplitude Vₒ′ of the applied voltage. The resulting modulation of the capacitance is C=C′/(1+δ sin ω′t) with C′=Kε₀A/d₀. In the latter equation, K is the average value of the dielectric constant of the materials between the plates (K=1 for air), ε₀ is the permittivity constant of the vacuum and A is the surface of one of the plates of the parallel plate capacitor.

The active capacitively coupled electrode with the variable capacitance of this invention can be secured to a living body in many different ways depending on the application. For EEG measurements, the best way to secure the electrodes in place on the scalp is to use a helmet. The electrodes can be fixed tightly in holes corresponding to the exact location of the locations described in the 10-20 international system of EEG electrode placement. Monitoring EMG activity on a limb can be done using a stretch band stretching around the limb. The extremities of the band could be fixed together using the Velcro system. The same procedure using stretch bands can be used on the torso for EKG measurements, for example. In these cases, the electrode would be embedded in the tissue of the stretching band. Other methods of fixing the electrodes could include the use of tape or adhesives (on the limbs or the main body), using a holder arm firmly fixed to the patients' bed or chair or other furniture around her/him, etc.

In operation, a frequency source 17 provides a predetermined frequency, a sequence of predetermined frequencies or random frequencies which excite the piezoelectric crystal 43 so as to effect vibration of the piezoelectric crystal 43. Vibration of the piezoelectric crystal 43 varies the spacing of the first 40 and second 41 plates of the variable capacitance device 12.

Further according to one embodiment of the present invention, the detection circuit 7 merely comprises a resistor which develops a voltage drop across the two inputs to the amplifier 8.

The detection circuit 7 is in electrical communication with a reference electrode 20. The reference electrode 20 and/or the ground electrode 21 are preferably contemporary conductively coupled electrodes and are preferably coupled to a monitoring device such as an EEG monitor, an EMG monitor, an EKG monitor or a GSR monitor according to well-known principles. Alternatively, the reference electrode 20 and the ground electrode 21 are capacitively coupled electrodes formed according to the present invention and are coupled to the monitoring device in a manner analogous to coupling of the capacitively coupled electrode 10 thereto.

When used in the performance of an EEG, for example, then the reference electrode 20 is typically attached to a patient at a location close to the location of the capacitively coupled electrode 10, such as at the lobe of one ear. During EEG procedures the ground electrode is typically placed on the patient in a region of lowest electrical potential, such as a boney structure, typically the boney structure of the C-7 vertebra.

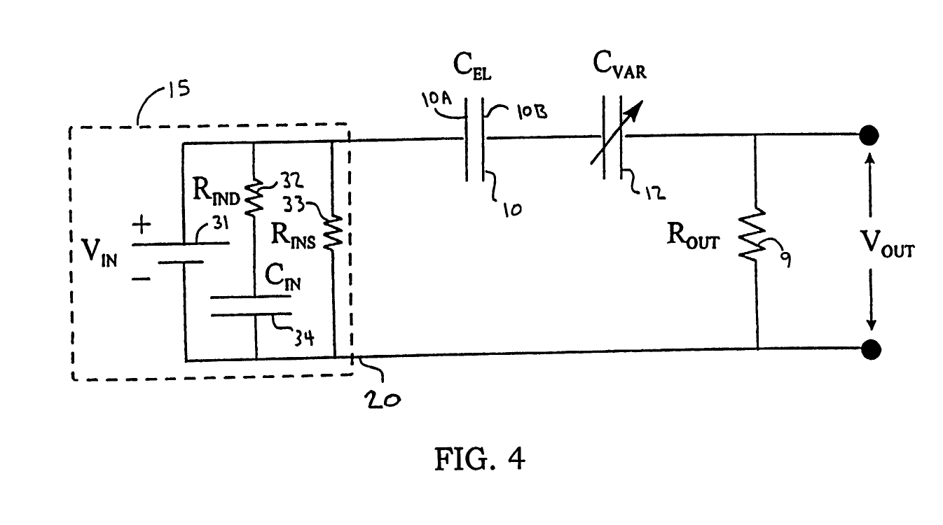

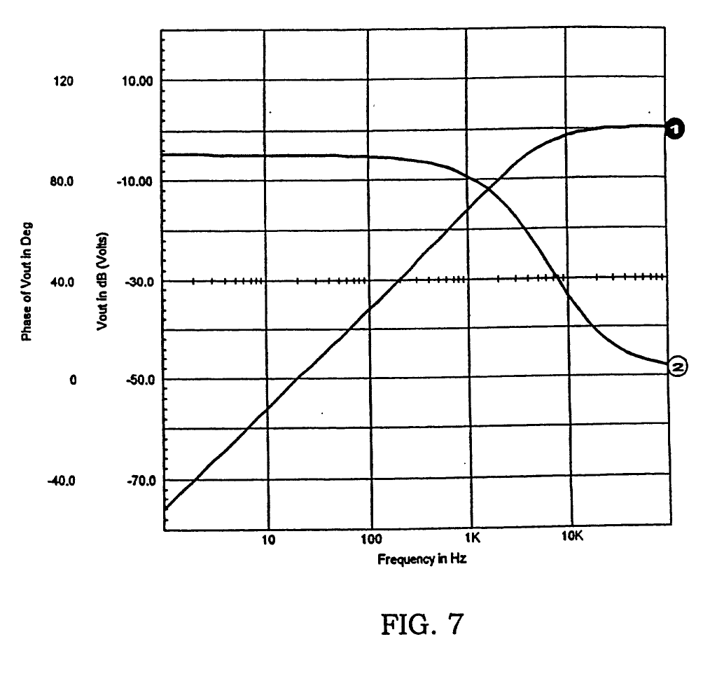

The amplifier 8 preferably comprises a variable gain differential amplifier, so as to facilitate adjustment of the amplitude of the signal output hereby. The variable gain differential amplifier provides a frequency dependent gain adjustment as a compensation for the frequency dependent transfer function of the electrode system as shown in the Bode diagram of FIG. 7. FIG. 7 shows a logarithmic dependence of the output voltage (Vₒᵤₜ in FIG. 4) with the frequency f of the input signal of the test item at low frequencies (f<10 kHz). This dependence is compensated by an inverse logarithmic dependence of the amplifier gain to be adjusted to the specific condition of each capacitive electrode of this invention. Additionally, the differential amplifier has a general gain to adjust the overall output voltage to match exactly the amplitude of the input voltage of the test item. Adjustment of the output of the amplifier 8 facilitates use of the capacitively coupled electrode system of the present invention in a variety of different applications, including but not limited to EEG, EMG, EKG and GSR applications. As those skilled in the art will appreciate, the electrodes utilized in each of these different procedures are generally different from one another, and therefore generally provide different output amplitudes. Thus, by adjusting the amplifier 8, an amplitude which is generally representative of the desired electrode, e.g., EEG electrode, EMG electrode, EKG electrode or a GSR electrode, can be provided.

Referring now to FIG. 4, a simplified schematic of the present invention shows the basic components thereof cooperating with a test subject is to provide an output signal (V_{OUT}). This simplified electrical schematic was used in a simulation to validate the desired operation of the present invention.

The test subject 15 is simulated with: a voltage source 31; a resistor 32 in series with a capacitor 34, both of which are in parallel with the voltage source 31; and a resistor 33 which is also in parallel with the voltage source 31. The voltage source 31 provides a varying input voltage V_{IN}. The resistor 32 has a resistance R_{IND} The capacitor 34 has a capacitance C_{IN}. The resistor 33 has a resistance R_{INS}.

The capacitively coupled electrode 10, in combination with the test subject 15, defines a capacitor which provides a capacitance C_{EL}. That is, the test subject 15 defines a first plate 10A of the capacitor and the capacitively coupled electrode 10 defines the second plate 10B thereof. In this manner, electrical activity within the test subject 15 is sensed as displacement current through the closed loop circuit formed by the subject's equivalent circuit and C_{EL}, C_{VAR} and R_{OUT}. Variable capacitance 12 provides a varying capacitance Cᵥₐᵣ. Output resistor 9 provides an output resistance R_{OUT} and is capacitively coupled with the test subject 15 via capacitively coupled electrode 10 and variable capacitance device 12 on one side thereof and is conductively coupled to the test subject 15 on the other side thereof via the reference electrode 20.

It can be seen that a closed loop circuit is formed by the test subject 15, the capacitively coupled electrode 10, the variable capacitance device 12, the resistor 9 and the reference electrode 20. If the variable capacitance device 12 is considered to be simply a parallel plate capacitor whose capacitance C_{VAR} is changed by a fast sinusoidal variation of the distance d between the capacitor plates such that d=d₀(1+δ sin(ω′t)), then Cᵥₐᵣ=Cᵥₐᵣ′/(1+δ sin(ω′t)) with Cᵥₐᵣ′=ε₀A/d₀. In the last two equations, d₀ is the distance between the two plates of the parallel plate capacitor at t=0 second, δ is the fraction of modulation of the capacitance of the variable capacitor (δ=1 represents 100% modulation; δ=0 represents no modulation), ω′=2πf′ with f′ the frequency of variation of the distance between the capacitor plates, ε₀ is the permittivity of a vacuum and A is the surface of one plate of the parallel plate capacitor.

Assuming that the detection circuit is a simple resistor, the closed loop circuit can be readily analyzed to give the voltage output V_{OUT} to be fed to the variable differential amplifier. The resulting circuit is presented in FIG. 4, along with the symbols representing the variables used in the mathematical analysis. For the purpose of this analysis, the living body is modeled as a skin surface resistor R_{INS} in parallel with a low frequency voltage source V_{IN} both in parallel with a capacitor C_{IN} in series with a dermis resistor R_{IND}.

The definition of the variables in FIG. 4 is as follows: V_{IN}=V sin ωt is the slowly varying voltage generated by the body between the capacitively coupled electrode and the reference electrode; R_{INS} is the electrical resistance of the epidermis between the capacitively coupled electrode and the reference electrode at the surface of the skin; C_{IN} is the capacitance of the body between the capacitively coupled electrode and the reference electrode mainly generated at the basal membrane (between the epidermis and the dermis); R_{IND} is the electrical resistance of the epidermis and dermis regions in series with C_{IN}; C_{EL} is the capacitance of the capacitively couple electrode; C_{VAR} is the capacitance of the variable capacitor; and R_{OUT} is the resistance of the detection resistor.

If the circuit components C_{EL}, C_{VAR} and R_{OUT} are chosen carefully, they can serve as a filter to filter out the high frequency component f′ of the variable capacitor (even if these components look placed to form a high pass filter). The statement will be justified below with the results of the simulations. In that case, one can average the high frequency components of the mathematical analysis and calculate an expression of the output voltage V_{OUT} which depends only on the low frequency f generated by the test item. The resulting formula for the voltage V_{OUT} across the detection resistor R_{OUT} is:

$$\begin{matrix}{V}_{OUT} = V \{ \frac{{ω}^{2} {R}_{OUT}^{2} {C}_{eq}^{2} cos ω t - ω {R}_{OUT} {C}_{eq} sin ω t}{1 + {ω}^{2} {R}_{OUT}^{2} {C}_{eq}^{2}} \} + \\ \text{small correction terms.}\end{matrix}$$

In the above equation C_{eq}=(C_{VAR}⁻¹+C_{EL}⁻¹)⁻¹ is the equivalent capacitance, ω=2πf, f is the frequency of oscillation of V_{IN} in cycles per second or Hz, π=3.1416. The equation for V_{OUT} above assumes a sinusoidal variation of the distance between the two plates of a parallel plate capacitor at the frequency f′=ω′/2π which is much larger than f=ω/2π. This sinusoidal variation is just one example of an infinite number of ways the capacitance of the variable capacitor can be varied. For example, the capacitance C_{VAR} could be varied by varying the permittivity of a dielectric material placed between the two plates such that ε=ε₀(1+δ sin ωt). Alternatively, the surface of the plates of C_{VAR} can be varied as A=A₀(1+δ sin ωt). Methods for varying the permittivity ε or the area A of the plates are well-known.

In order to check the validity of the above equation, a simulation of the closed loop circuit analyzed above was performed using a commercially available circuit simulation software. For the simulation purposes, the following parameter values were chosen:

V=2 μV

f′=ω′/2π=10,000 Hz

R_{IND}=1 kΩ

R_{INS}=100 kΩ

C_{IN}=40 nF

C_{EL}=3pF

C_{VAR}=1 μF

δ=0.5

f=ω/2π=1 Hz

R_{OUT}=10 MEGΩ

These parameters were chosen to simulate an EEG signal at the input and to provide the highest output signal possible without any distortion.

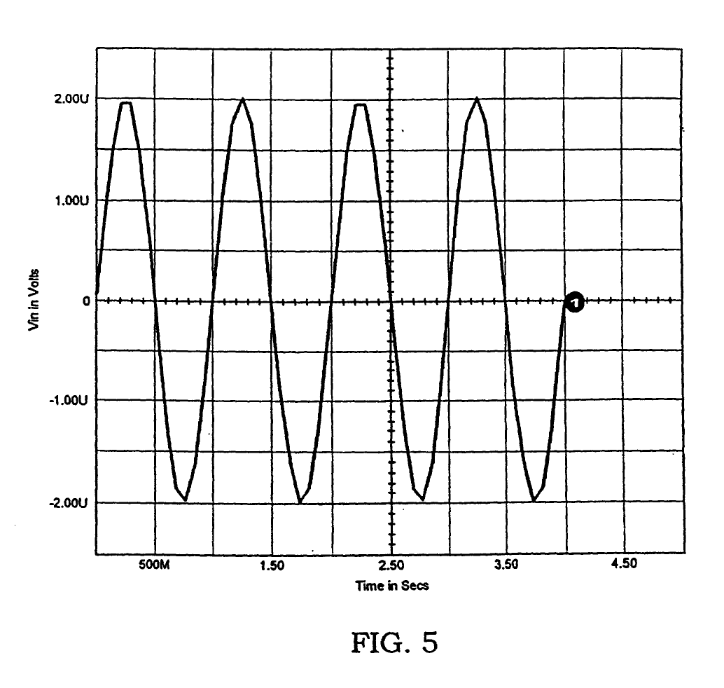

FIG. 5 presents the generally sinusoidal input signal V_{IN}=V sin ωt.

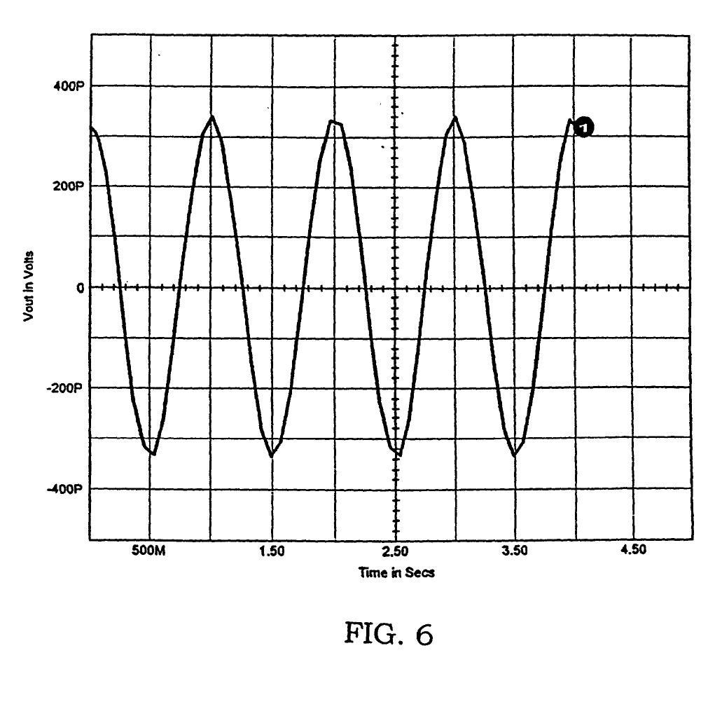

FIG. 6 presents the generally sinusoidal output voltage V_{OUT}. With the values chosen above ωR_{OUT}C_{eq}=1.88×10⁻⁴<<1 and the maximum amplitude of V_{OUT,}|V_{OUT}|ₘₐₓ is |V_{OUT}|ₘₐₓ=VωR_{OUT}C_{eq}=3.77×10⁻¹⁰ cos ωt, in good agreement with the simulation shown in FIG. 6.

FIG. 7 presents a Bode diagram (output voltage and phase vs. frequency) for the simulation parameters described above. One may note the saturation of the output voltage above f′=10,000 Hz. The equation for V_{OUT} shows that the output voltage should be independent of the frequency of modulation of the capacitor f′ and the fraction of modulation of the capacitance δ. V_{OUT} should also be independent of Cᵥₐᵣ′ as long as Cᵥₐᵣ′>>C_{EL}. The independence of the output voltage on f′ is apparent in FIG. 6, as no high frequency modulation signal is observed. This result justify our assumption to average the high frequency terms that are generated by the variable capacitor as mentioned previously when calculating the output voltage V_{OUT}. Additional simulations showed that there were no change in V_{OUT} for 0.1<δ<0.9 and when Cᵥₐᵣ′>>C_{EL}. More simulations showed that the linear dependency of V_{OUT} on ω, R_{OUT} and C_{eq} is valid as long as ωR_{OUT}C_{eq}<<1 and Cᵥₐᵣ′>>C_{EL}.

The presence of the variable capacitance is not only desirable, but is important for the electrode to function as described. The variable capacitance generates the displacement current without which there is no current in the circuit comprised of the electrode, the variable capacitor and the detection circuit. For the clarity of the discussion here, let us call the circuit mentioned in the last sentence the electrode circuit. The electrical potential generated by the test item is generally too weak to generate any current in the electrode circuit (especially in the case of EEG). Without a current in the electrode circuit, there is no means to recover the potential generated by the test item (unless we use resistively coupled electrodes which is what we are trying to avoid with this invention).

The goal of the electrode of this invention is to monitor the electrical potential generated at the surface of the tissue of the test item without distortion and without the use of a resistively coupled electrode. This is accomplished by capacitively coupling the electrode to the test item and by generating a variable current in the electrode circuit.

There are two other ways we know to generate a variable current in the electrode circuit. These are: to include in the electrode circuit a variable voltage source or to include in the electrode circuit a variable current source. There are problems with both methods. The problem with adding a variable voltage source is that this variable voltage is added to the very small potential generated at the surface of the skull (in the case of EEG, for example). To separate these two voltages accurately would require complex electronic circuits because they are so small (in the microvolt range for EEG). The problem with adding a current source is that the voltage at the detection circuit includes an amplitude modulation (AM) of the potential generated by the test item and the voltage generated in the electrode circuit by the variable current source. This is similar to AM modulation used for radio transmission. This would need an AM demodulator, a complex circuit for such a would-be simple electrode. The variable capacitor eliminates these problems.

Electric circuit theory and electrical simulations using a commercially available software showed that if the variable capacitor is varied at a frequency that is at least 10 times the maximum frequency expected to be generated by the test item, then there is a possibility to eliminate the effect of this rapidly varying capacitor simply by choosing the components of the electrode circuit in such a manner that this circuit act like a filter which filter out high frequency components and leave intact the low frequency components generated by the test item.

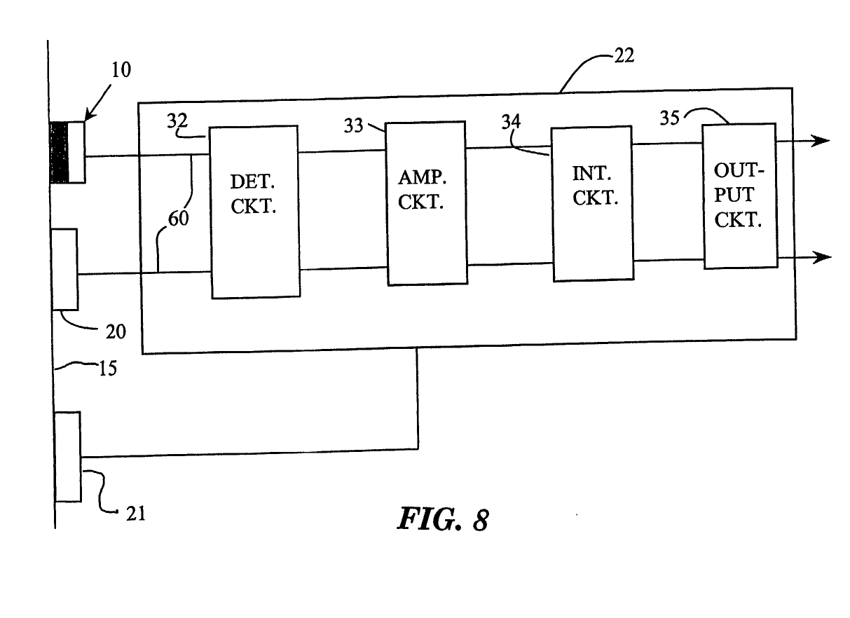

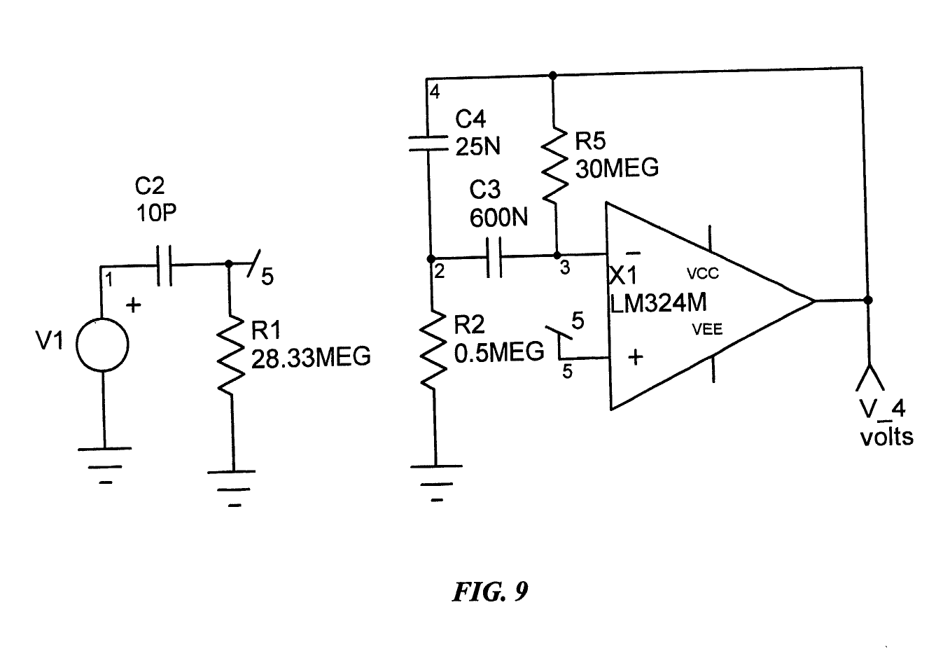

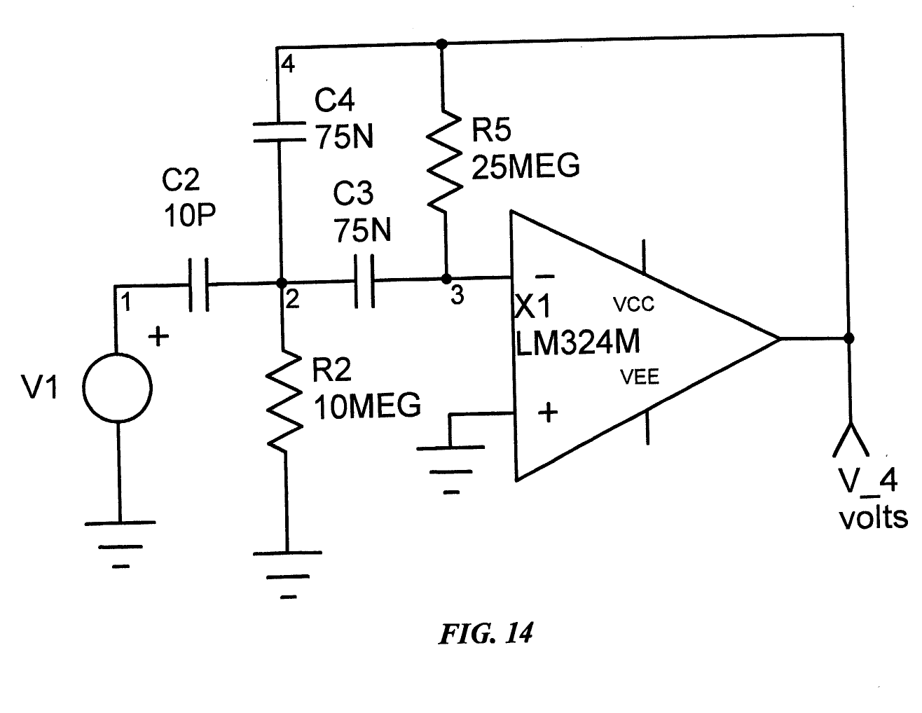

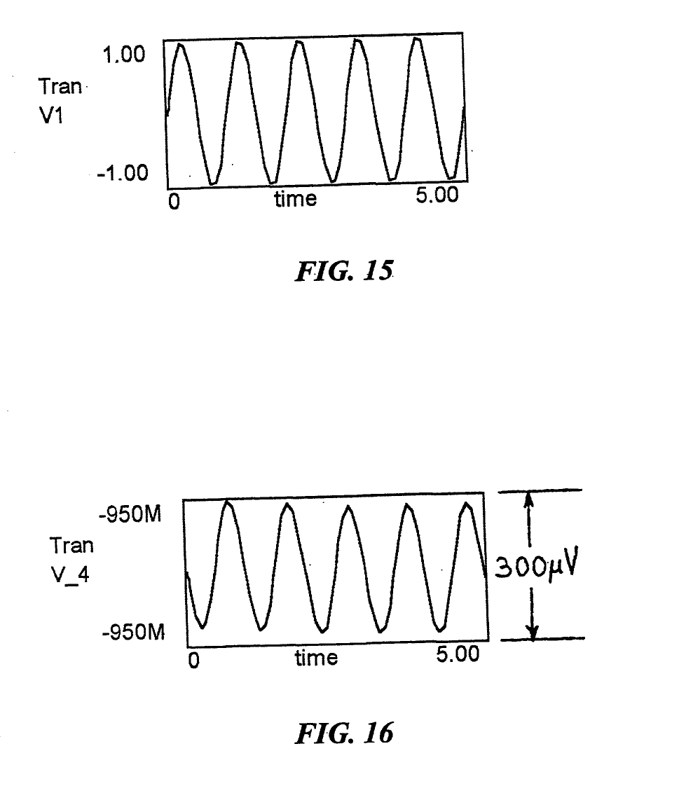

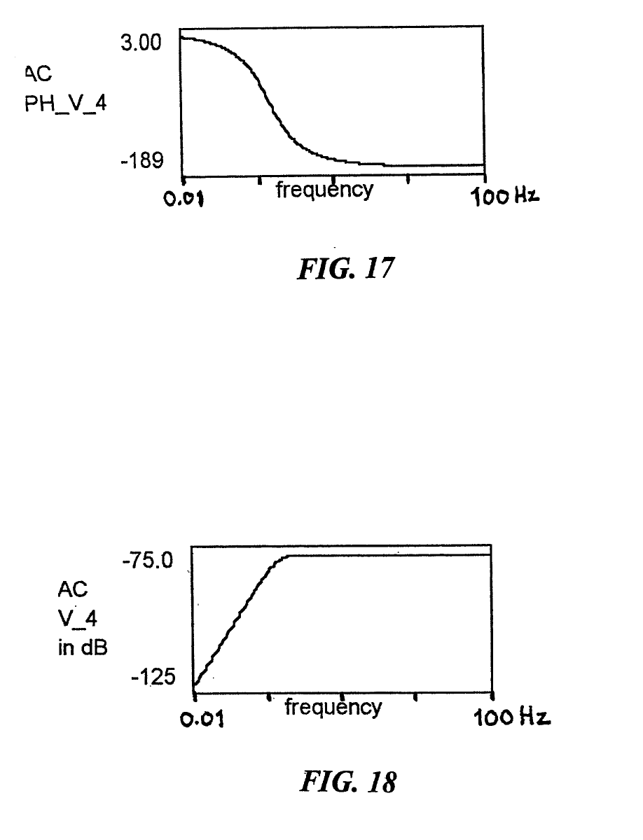

FIG. 8 shows a block diagram common to both the second and third embodiments of the present invention. FIGS. 9-13 show a detection circuit of second embodiment of the capacitively coupled electrode system of the present invention and data obtained from a computer simulation thereof. FIGS. 14-18 show a detection circuit of third embodiment of the present invention and data obtained from the computer simulation thereof. According to both the second and third embodiments of the present invention, the variable capacitance device, along with the frequency generator associated therewith, are eliminated so as to simplify construction and use of the invention.

According to the second and third embodiments of the present invention, the detection circuit receives the output of the capacitively coupled electrode 10. The detection circuit 32 also receives the output of the reference electrode 20. Preferably, the detection circuit 32, along with the amplification circuit 33, the integration circuit 34 and the output circuit 35, are housed within a common conductive container, such as a metal box 22, which defines a chassis ground. Ground electrode 21, which is attached to the test subject 15 assures that the test subject 15 and the chassis ground are at the same potential, so as to prevent shock to the patient and/or medical personnel.

The detection circuit 32 is specifically configured so as to mitigate the effects to the output of the capacitively coupled electrode 10 which are due to capacitive coupling thereof (as oppose to direct electrical or conductive coupling thereof). That is, the detection circuit 32 tends to modify the electrical output of the capacitively couple electrode 10 in a manner which makes the output thereof more like the output of a direct electrically or conductively coupled electrode. In mitigating the effects of capacitive coupling of the electrode 10, the detector circuit 32 differentiates the signal. An integration circuit 34 integrates the output of the detectors circuit so as to substantially cancel the effects of differentiation thereof, as described below.

With particular reference to FIG. 8, a capacitively coupled electrode 10, reference electrode 20, and a ground electrode 21 are placed at the surface of the skin 15 of a test subject. As with the first embodiment of the present invention, the test subject 15, may be mammalian tissue, or may alternatively be any other desired test subject. Frequently, the test subject will be a person who is undergoing a medical procedure such as an electroencephalograph (EEG), an electromyograph (EMG), an electrocardiograph (EKG) or a galvanic skin response (GSR) test. The capacitively coupled electrode 10, referenced electrode 20, and ground electrode 21 may, if desired, be identical to those discussed above for use with the first embodiment of the present invention. The capacitively coupled electrode 10, the referenced electrode 20 and the ground electrode 21, perform the same functions in the second and third embodiment of the present invention as they performed in the first embodiment thereof.

The detection circuit 32 is specifically configured so as to substantially cancel the capacitive effect of the capacitively coupled electrode 10. That is, the detection circuit 32 tends to modify the output of the capacitively coupled electrode 10 in a manner in which make the output of the capacitively coupled electrode 10 more like the output of a direct electrically couple electrode. The amplification circuit 33 amplifies the output of the detection circuit 32 by a factor preferably ranging from approximately 30 to approximately 7,000. The amplification of the amplification circuit 33 is depending upon the specific detection circuit utilized, as well as the desired application of the present invention. Generally, the amplification circuit 33 will provide amplification which approximately compensates for loss in signal strength caused by the detection circuit 32.

The integration circuit 34 integrates the output of the amplification circuit 33 in a manner in which tends to compensate for the differentiation caused by the detection circuit 32. Integration circuits are well known in the art.

The output circuit 35 adjusts the output impedance of the present invention so as to provide an output impedance which is compatible with the desired next stage of processing, such as that provided by an EEG, EMG, or EKG apparatus.

Referring now to FIG. 9, one preferred configuration of the detection circuit 32 defines an exemplary second embodiment of the present invention. As shown in FIG. 9 (and as shown in FIG. 14, as well) the capacitively coupled electrode 10 (C2 in FIG. 9) is coupled to the detection circuit 32 at node 5, while the reference electrode 20 is coupled to the detection circuit 32 at the grounds shown in FIG. 9. Thus, the voltage V1 provided by the test subject 15 is generated between the capacitively couple electrode 10 and the reference electrode 20, as shown in FIG. 9.

According to the second embodiment of the present invention, a LM324M operational amplifier (X1) has two capacitors (C3 and C4) and three resistors (R1, R2 and R5) whose values have been specifically selected so as to substantially cancel the effects of capacitively coupling the capacitively coupled electrode 10 to the test subject, rather than direct electrically coupling the electrode thereto. Thus, according to the second embodiment of the present invention the value of C3 is 600 nF, the value of C4 is 25 nF, the value of R1 is 28.33 megohm, the value of R2 is 0.5 megohm and the value of R5 is 30 megohm.

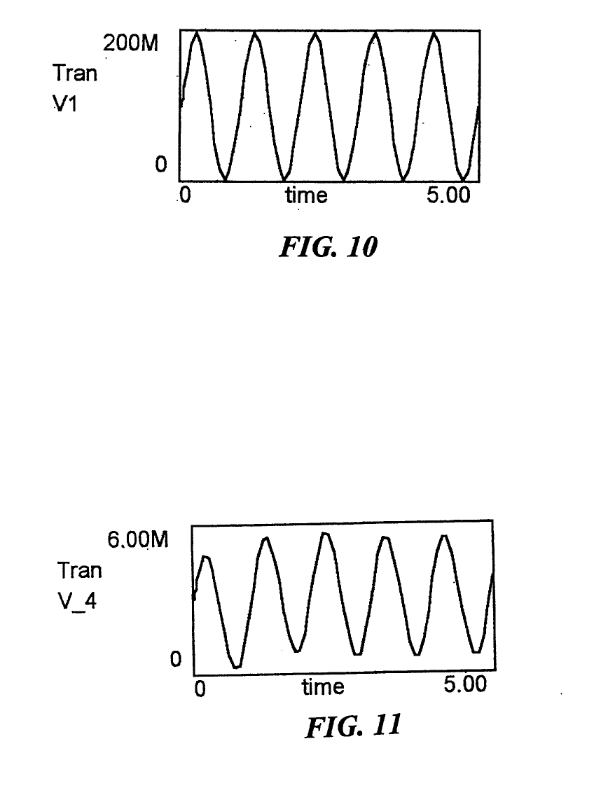

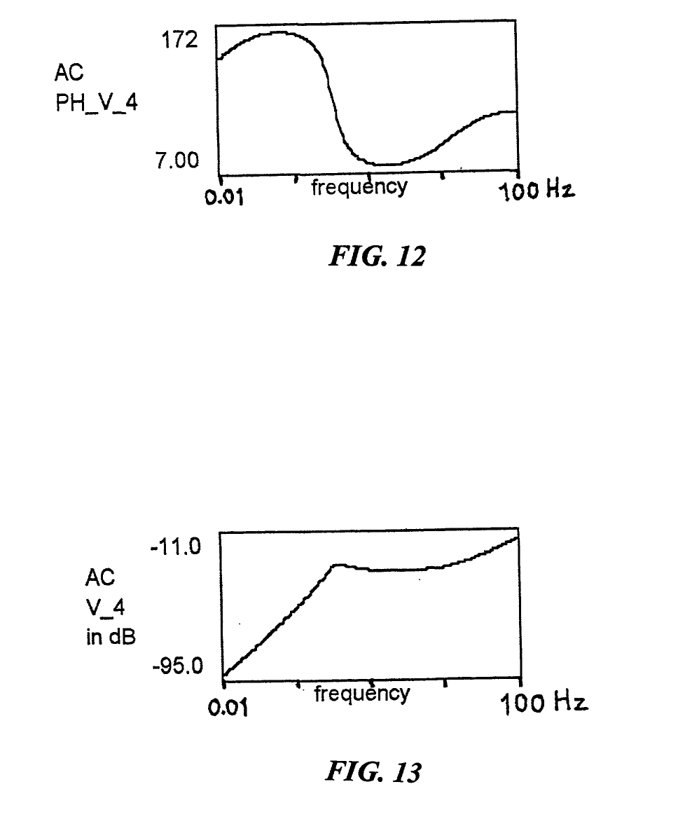

A computer simulation of the detection circuit 32 of FIG. 9, using the input voltage shown in FIG. 10 provides the output voltage shown in FIG. 11. Phase variation is shown in FIG. 12 and the frequency response is shown in FIG. 13.

As mentioned above, V1 represents the electrical potential generated by the test subject between the capacitively coupled electrode 10 and the reference electrode 20, C2 represents the capacitance of the capacitively coupled electrode 10. That is, C2 is the capacitance defined by the interface of the capacitively coupled electrode 10 and the test subject 15, wherein the capacitively coupled electrode 10 itself defines one plate of a capacitor, the test subject 15 defines the other plate thereof and the interface there between defines the dielectric 14 thereof. C2 is estimated to be approximately 10 pF. The remaining capacitors and resistors of FIG. 9 are part of the detection circuit 32.

The operational amplifiers LM324M (X1) is one example of an amplifier that can be used in the detection circuit 32 of the present invention. As can be seen from the computer simulation data shown in FIGS. 10-13, an input voltage of 200 millivolts (FIG. 10) provides an output voltage of only 6.0 millivolts, peaks-2-peak (FIG. 11). As can be seen in FIG. 13, the frequency response is substantially flat between 0.1 Hz and 100 Hz, thus substantially mitigating the need for frequency compensation of the detection circuit 32.

With particular reference to FIGS. 14-18, a third embodiment of the present invention is provided. According to the detection circuit 32 of the third embodiment of the present invention, the values of the two capacitors (C3 and C4) and the two resistors (R2 and R5) are different with respect to the second embodiment of the present invention (the resistor R1 has been eliminated). According to the third embodiment of the present invention, the capacitor C3 has a value of 75 nF, capacitor C4 as a value of 75 nF, resistor R2 has a value of 10 megohm, and resistor R5 has a value of 25 megohm.

The detection circuits of the second (FIG. 9) and third (FIG. 14) embodiments, are two representative examples of suitable detection circuits that can be obtained by manipulation of the passive (resistors and capacitors) an active (operational amplifier) components. Those skilled in the art will appreciate that various combinations of such components likewise suitable for providing a detection circuit which substantially mitigates the effects of capacitively coupling an electrode to a test subject and are thus equivalent to the detection circuit, of FIGS. 9 and 14.

The detention circuit 32 of the second embodiment of the present invention does not invert the input signal provided thereto. Further, the detection circuit 32 of the second embodiment of the present invention does not decrease the input signal by as much as detection circuit 32 of the third embodiment thereof. The detection circuit 32 of the second embodiment of the present invention decreases the input signal by a factor approximately 33.3, whereas the detection circuit 32 of the third embodiment of the present invention reduces the input signal by a factor of approximately 7,000. According to the detection circuit 32 of the second embodiment of the present invention, the output voltage is almost constant between 0.3 Hz and 100 Hz.

It is understood that the exemplary capacitively coupled electrode system described herein and shown in the drawings represents only a presently preferred embodiment of the invention. Indeed, various modifications and additions may be made to such embodiment without departing from the spirit and scope of the invention. For example, various different configurations of the electrode and/or variable capacitance device are contemplated. Thus, these and other modifications and additions may be obvious to those skilled in the art and may be implemented to adapt the present invention for use in a variety of different applications.

1. An electrical activity sensor system comprising:
an electrode configured to be capacitively coupled to a test item;
a detection circuit configured to mitigate a capacitive effect of the electrode; and
wherein the capacitively coupled electrode and the detecting circuit cooperate to mitigate a need for conductively coupling an electrode to the test item.

2. The electrical activity sensor system as recited in claim 1, wherein the electrode comprises:
a conductive member; and
a dielectric member configured to inhibit contact of the conductive member with the test item.

3. The electrical activity sensor system as recited in claim 1, wherein the electrode comprises:
a conductive member generally configured as a disk; and
a dielectric cover substantially surrounding the conductive member.

4. The electrical activity sensor system as recited in claim 1, wherein the electrode is configured to be capacitively coupled to living tissue.

5. The electrical activity sensor system as recited in claim 1, wherein the electrode is configured to be capacitively coupled to a mammal.

6. The electrical activity sensor system as recited in claim 1, wherein the electrode is configured to be capacitively coupled to a human being.

7. The electrical activity sensor system as recited in claim 1, wherein the electrode comprises:
a copper member generally configured as a disk;
a dielectric cover substantially surrounding the conductive member;
a cap comprised of insulator cooperating with the dielectric cover to generally enclose the copper member; and
a conductive lead coupled to the copper member and extending through the cap.

8. The electrical activity sensor system as recited in claim 1, wherein the detection circuit comprises an amplifier circuit which is configured to mitigate a capacitive effect of the electrode.

9. The electrical activity sensor as recited in claim 1, wherein the detection circuit comprises:
an operational amplifier; and
at least one resistor and at least one capacitor in electrical communication with the operational amplifier.

10. The electrical activity sensor system as recited in claim 1, wherein the detection circuit comprises:
an operational amplifier; and
two resistors and two capacitors in electrical communication with the operational amplifier.

11. The electrical activity sensor system as recited in claim 1, wherein the detection comprises:
an operational amplifier having a gain of approximately 1; and
wherein the operational amplifier is in electrical communication with circuitry which cooperates with the operational amplifier to mitigate a capacitive effect of the electrode.

12. The electrical activity sensor system as recited in claim 1, wherein the detection circuit comprises:
a LM324M operational amplifier;
a 25 nF capacitor in electrical communication with the operational amplifier;
a 600 nF capacitor in electrical communication with the operational amplifier;
a 30 megohm resistor in electrical communication with the operational amplifier;
a 0.5 megohm resistor in electrical communication with the operational amplifier; and
a 28.33 megohm resistor in electrical communication with the operational amplifier.

13. The electrical activity sensor system as recited in claim 1, wherein the detection circuit comprises:
a LM324M operational amplifier;
a first 75 nF capacitor in electrical communication with the operational amplifier;
a second 75 nF capacitors in electrical communication with the operational amplifier;
a 25 megohm resistor in electrical communication with the operational amplifier; and
a 10 megohm resistor in electrical communication with the operational amplifier.

14. The electrical activity sensor system as recited in claim 1, wherein the detection circuit comprises a LM324M operational amplifier configured to have one input thereto in electrical communication with the capacitively coupled electrode, and to have the other input thereto in electrical communication with a reference electrode.

15. The electrical activity sensor system as recited in claim 1, further comprising an amplification circuit coupled to receive an output of the detection circuit.

16. The electrical activity sensor as recited in claim 1, further comprising an integration circuit coupled to receive an output of an amplification circuit, the amplification circuit being coupled to receive an output of the detection circuit.

17. The electrical activity sensor system as recited in claim 1, further comprising an integration circuit coupled to receive an output of an amplification circuit, the amplification circuit being couple to receive an output of the detection circuit and the detection circuit differentiating an output of the capacitively coupled electrode.

18. The electrical activity sensor system as recited in claim 1, further comprising:
an amplifier coupled to amplify an output of the detection circuit; and
wherein the amplifier has a gain of between 6 and 7,000.

19. The electrical activity sensor system as recited in claim 1, further comprising:
a variable gain amplifier coupled to amplify an output of the detection circuit in a manner which facilitates provision of an output that generally mimics an output of at least one of an electroencephalograph electrode, an electrocardiograph electrode, an electromyograph electrode and a galvanic skin response electrode.

20. The electrical activity sensor as recited in claim 1, further comprising:
an amplifier coupled to amplify an output of the detection circuit;
an integration circuit coupled to integrate an output of the amplifier circuit; and
an output circuit coupled to the amplifier to define an output impedance.

21. The electrical activity sensor system as recited in claim 1, further comprising:
an amplifier coupled to amplify an output of the detection circuit; and
an output circuit coupled to the amplifier to define an output impedance which is suitable for providing a signal to an electroencephalograph.

22. The electrical activity sensor system as recited in claim 1, further comprising;
an amplifier coupled to amplify an output of the detection circuit; and
an output circuit coupled to the amplifier to define an output impedance which is suitable for providing a signal to an electromyograph.

23. The electrical activity sensor system as recited in claim 1, further comprising:
an amplifier coupled to amplify an output of the detection circuit; and
and output circuit coupled to the amplifier to define an output impedance which is suitable for providing a signal to an electrocardiograph.

24. The electrical activity sensor system as recited in claim 1, further comprising:
an amplifier coupled to amplify an output of the detection circuit; and
an output circuit coupled to the amplifier to define an output impedance which is suitable for providing a signal to a galvanic skin response monitor.

25. The electrical activity sensor system as recited in claim 1, further comprising a reference electrode coupled to the detection circuit.

26. The electrical activity sensor system as recited in claim 1, further comprising a ground electrode coupled to a metal enclosure, the metal enclosure enclosing the detection circuit.

27. The electrical activity sensor system as recited in claim 1, further comprising a reference electrode coupled to the detection circuit and a ground electrode coupled to a metal enclosure.

28. The electrical activity sensor system as recited in claim 1, further comprising a capacitively coupled electrode coupled to a detection circuit which differentiates an output of the capacitively coupled electrode and an integrated circuit coupled to integrate an output of the detecting circuit.

29. A method for characterizing electrical activity of an object being monitored, the method of comprising using displacement current to sense electrical activity within the test item, differentiating a signal representation of the displacement current, and integrating the signal representative of the displacement current.

30. The method as recited in claim 29, wherein using displacement current to sense electrical activity comprises capacitively coupling an electrode to the object being tested.

31. The method as recited in claim 31, wherein using displacement current to sense electrical activity comprises capacitively coupling an electrode to the object being tested, differentiating an output of a capacitively coupled electrode and integrating the output of the capacitively coupled electrode.
